(19) United States
(12) Patent Application Publication
(10) Pub. No.: US 2009/0011431 A1
(43) Pub. Date: Jan. 8, 2009

(54) Diagnosis of Sepsis by the Selective Determination of the Concentration of Cu/Zn Superoxide Dismutase (Cu/Zn Sod) in Patient Samples

(75) Inventors: Andreas Bergmann, Berlin (DE); Joachim Struck, Berlin (DE); Monika Uhlein, Berlin (DE); Nils G. Morgenthaler, Berlin (DE)

Correspondence Address: HESLIN ROTHENBERG FARLEY & MESITI PC, 5 COLUMBIA CIRCLE, ALBANY, NY, 12203, US

(73) Assignee: B.R.A.H.M.S AKTIENGESELLSCHAFT, Hennigsdorf (DE)

(21) Appl. No.: 10/597,619

(22) PCT Filed: Feb. 2, 2005
(86) PCT No.: PCT/EP2005/001037

§ 371 Date: Sep. 9, 2008

(30) Foreign Application Priority Data
Feb. 3, 2004 (EP) 04002355.8

(51) Int. Cl. G01N 33/573

(52) U.S. Cl. 435/7.4

(57) ABSTRACT

The present invention relates to a method for the early determination of the risk of mortality of patients in intensive care units or emergency care units during which the concentration of Cu/Zn superoxide dismutase (Cu/Zn SOD) in a serum sample or plasma sample of the patient is selectively determined, and quantitatively or semi-quantitatively measured concentrations, which exceed a predetermined threshold value are correlated with a high risk of mortality.

### BACKGROUND OF THE INVENTION

The present invention relates to a novel prognostic method which can be used in the clinical care of patients in intensive care units and emergency care units, in particular sepsis patients, and provides information about the risk of mortality or about the probability of survival of such patients.

The present invention has a starting point in intensive research work by the Applicant in relation to further improvements of the diagnosis and therapy of sepsis.

Inflammations are defined very generally as certain physiological reactions of an organism to a variety of external effects, such as, for example, injuries, burns, allergens, infections by micro-organisms, such as bacteria and fungi and viruses, to foreign tissues which trigger rejection reactions, or to certain inflammation-inducing endogenous conditions of the body, for example in autoimmune diseases and cancer. Inflammations may occur as harmless, localised reactions of the body but are also typical features of numerous serious chronic and acute diseases of individual tissues, organs, organ parts and tissue parts.

Local inflammations are generally part of the healthy immune reaction of the body to harmful effects and hence part of the life-preserving defense mechanism of the organism. However, if inflammations are part of a misdirected reaction of the body to certain endogenous processes, such as, for example, in autoimmune diseases and/or are chronic in nature, or if they reach systemic levels, as in systemic inflammatory response syndrome (SIRS) or in a severe sepsis caused by infection, the physiological processes typical for inflammation reactions grow out of control and become the actual, frequently life-threatening pathological process of patients, who accordingly typically have to be cared for in intensive care units of a hospital. In the following description, such patients are referred to as a rule as sepsis patients, but this term does not necessarily imply a prior sepsis diagnosis of the patients and is intended generally to include critically ill patients in intensive care units for whom there is a risk of sepsis.

It is now known that the origin and the course of inflammatory processes are controlled by a considerable number of substances which are predominantly of a protein or peptide nature or are accompanied by the occurrence of certain biomolecules for a more or less limited time. The endogenous substances involved in inflammatory reactions include in particular those which can be included among the cytokines, mediators, vasoactive substances, acute phase proteins and/or hormonal regulators. The inflammatory reaction is a complex physiological reaction in which both endogenous substances (e.g. TNF-α) activating the inflammatory process and deactivating substances (e.g. interleukin-10) are involved.

In systemic inflammations, as in the case of a sepsis or of septic shock, the inflammation specific reaction cascades spread in an uncontrolled manner over the whole body and become life-threatening in the sense of an excessive immune response. Regarding the present knowledge about the occurrence and the possible role of individual groups of endogenous inflammation-specific substances, reference is made, for example, to A. Beishuizen et al., “Endogenous Mediators in Sepsis and Septic Shock”, Advances in Clinical Chemistry, vol. 33, 1999, 55-131; and C. Gabay et al., “Acute Phase Proteins and Other Systemic Responses to Inflammation”, The New England Journal of Medicine, vol. 340, no. 6, 1999, 448-454. Since the understanding of sepsis, and hence also the recognised definitions, have changed in recent years, reference is also made to K. Reinhart et al., “Sepsis und septischer Schock” [Sepsis and septic shock], in: Intesivmedizin, Georg Thieme Verlag, Stuttgart, N.Y., 2001, 756-760, where a modern definition of the term sepsis is given. Regarding the importance of the clinical picture of “severe sepsis”, reference is furthermore made to (34; in this description, numbers in brackets refer to the references which appear under the same number in the list of references at the end of the description). A more recent summary of the criteria and definitions for a sepsis and closely related clinical pictures are to be found under http://www.talessin.de/scripte/medizin/sepsis1.html. In the present Application, the term sepsis is used in a comprehensive sense based on the definitions as they appear in said publications for septic clinical pictures of severely ill patients in intensive care units.

While at least in the European region systemic bacterial infection detectable by a positive blood culture long characterized the term sepsis, sepsis is now understood primarily as being systemic inflammation caused by infection but which, as a pathological process, has great similarities with systemic inflammation which have other causes. Said change in the understanding of sepsis is based on changes in the diagnostic approaches. Thus, the direct detection of bacterial pathogens has been replaced or supplemented by complex monitoring of laboratory parameters and hemodynamic parameters with the use of computer-aided so-called score systems (e.g. APACHE II SCORE; APACHE stands for “Acute Physiology and Chronic Health Evaluation”; cf. (33) and the introduction of DE 42 27 454 C1) and more recently in particular by the detection of certain endogenous substances involved in the sepsis process or in the inflammatory process, i.e. specific “biomarkers”.

Among the large number of mediators and acute phase proteins in particular those whose occurrence is very specific for sepsis or certain phases of a sepsis and whose concentrations change drastically and diagnostically significantly and which moreover have the stabilities required for routine determinations and reach high concentration value are suitable for diagnostic purposes. The reliable correlation of pathological process (sepsis) with the respective biomarker is of primary importance for diagnostic purposes, without it being necessary for its role in the complex cascade of endogenous substances involved in the sepsis process always to be known specifically.

Such an endogenous substance which is particularly suitable as a sepsis biomarker is procalcitonin. Procalcitonin is a prohormone whose serum concentrations reach very high values under the conditions of a systemic inflammation or infectious etiology (sepsis) whereas it is virtually undetectable in healthy persons. High values of procalcitonin are moreover reached in a relatively early stage of a sepsis so that the determination of procalcitonin is also suitable for the early diagnosis of a sepsis and for early distinction between a sepsis due to infection and severe inflammations which have other causes. The determination of procalcitonin as a sepsis marker is the subject of the publication by M. Assicot et al., “High serum procalcitonin concentrations in patients with sepsis and infection”, The Lancet, vol. 341, no. 8844, 1993, 515-518; and the patents DE 42 27 454 C2 and EP 0 656 121 B1 and U.S. Pat. No. 5,639,617. Reference is made expressly to said patents and to early literature references mentioned in said publication supplementing the present description.

The availability of the sepsis marker procalcitonin has given considerable impetus to sepsis research, and intensive efforts are currently being made to find further biomarkers which can supplement the procalcitonin determination and/or can provide additional information for the purposes of precise diagnosis or differential diagnosis. The search for potential novel sepsis biomarkers, however, is complicated by the fact that often very little or nothing is known about the exact function or about the exact reasons for the occurrence of certain endogenous substances which are involved in the sepsis process.

The results of the experimental testing of a fruitful, purely hypothetical approach to the determination of further potential sepsis markers are to be found in DE 198 47 690 A1 or WO 00/22439. There, it is shown that, in sepsis, not only the concentration of the prohormone procalcitonin is increased but significantly increased concentrations can also be observed for other substances which can be counted among the peptide prohormones or which are the fragments of such prohormones and have an immunoreactivity typical of such prohormones. While the phenomenon described is well documented, exact causes of the detectable increase of the concentrations of prohormones or their fragments in sepsis are substantially unexplained in most cases.

The present Application is based on a result of another fruitful, purely experimental approach in the search for further sepsis-specific biomolecules. This is based on the fact that, by administering an endotoxin to primates (baboons), an artificial sepsis is produced in them and endogenous substances of a peptide or a protein nature which are found only in the “septic” baboons and which therefore represent potential sepsis-specific biomarkers are determined by comparison of the gel electrophoresis protein spot samples of endotoxin-treated and untreated baboons. The primate model was chosen owing to the very great similarity of the physiology of primates and humans and the high cross-reactivity with many therapeutic and diagnostic human reagents.

As described more exactly in the experimental section of prior patent applications of the Applicant, a number of protein spots identifiable only in the treated animals is found after experimental induction of an artificial sepsis in baboons by endotoxin administration (LPS from Salmonella Typhimurium) and working-up of liver tissue of the treated animals by 2D gel electrophoresis. The protein products corresponding to the spots are isolated from the electrophoresis gel and investigated by mass spectrometry (especially by means of tandem mass spectrometry).

Inter alia, the proteins “inflammin” (WO 02/085937), CHP (WO 03/005035), soluble cytokeratin-1 fragments (sCY1F; WO 03/002600), the protein LASP-1 (WO 03/089934) and enzymes such as aldose-1-epimerase (mutarotase; WO 03/048780), glycine N-acyl transferase (GNAT; WO 03/048781) and soluble carbamoyl phosphate synthetase 1 (CPS 1; WO 03/089933) were identified as novel sepsis markers by said method, as described for the first time in prior German and European patent applications of the Applicant.

The content of said prior applications of the Applicant is to be regarded as part of the disclosure of the present Application by the express reference to these Applications.

The investigations described also revealed, as sepsis-specific protein spots, numerous substances which were already known as so-called “acute phase proteins” or for which a subsequent literature search showed that their occurrence in association with inflammations or sepsis had already been discussed.

Such a protein spot was also the spot which, when it was worked up analytically, proved to be a superoxide dismutase enzyme, namely the enzyme Cu— and Zn— dependent superoxide dismutase (Cu/Zn SOD; SOD-1).

Superoxide dismutases (SOD, EC 1.15.1.1) are enzymes having an antioxidant function which are capable of converting the reactive superoxide anion O₂⁻ into less reactive species. Eukaryotic cells contain two different SOD types, namely Cu/Zn SOD (also known as SOD-1) and Mn SOD. Human Cu/Zn SOD is primarily found in cytosol. Cu/Zn is a dimer consisting of two identical subunits and having a molar mass of about 33 kDa. Human Mn SOD (SOD-2), which is found in particular in the mitochondria, is a homotetramer and has a molar mass of about 80 kDa. In addition, a so-called extra cellular SOD or EC-SOD, which occurs inter alia in extra cellular fluids, such as plasma, lymph and synovial fluid, was also identified. The cDNA or amino acid sequences of all three abovementioned SOD types are known (cf. for example (27), (28), (29)) and differ considerably. They can be found in relevant databases (e.g. http://www.expasy.org/cgi-bin-niceprot) (Cu/Zn SOD or SOD1 Swiss-Prot Accession Number: P00441, Mn SOD or SOD2: Swiss-Prot Accession Number: P04179; extra cellular SOD or EC-SOD or SOD3; Swiss-Prot Accession Number: P08294). When only the abbreviation SOD is used in the following description, no distinction is made between the individual SOD types, i.e. the discussion is as a rule about findings and information where the enzymatic action is of primary importance.

Owing to the different sequences of their subunits, all three SOD types have different immunoreactivities and can be selectively determined with the aid of immunoassays which do not cross-react (cf. for example (2)). Both human Cu/Zn SOD and human Mn SOD were prepared in recombinant form for therapeutic purposes/experiments (cf. (5) and the further literature references cited therein).

There is in particular extensive scientific literature about SOD-1 and SOD-2, which however gives a complex and partly contradictory picture of the physiological occurrence and the role of the various SOD types in various pathological conditions, in various species and organs or tissues and under various external influences. Inter alia, it has long been known that the expression or translation of SOD can be influenced by endotoxins (LPS), and that the action of LPS, at least in rats, is dependent inter alia on the oxygen fraction of the respiratory air (cf. for example (1) and (3)). However, the experimental findings differ considerably depending on the origin of the tissue investigated or of the cell culture investigated and depending on whether the mRNA or the protein level is considered, the time interval of the observations also playing an additional role (cf. for example (1) to (4); (6), (7), (9), (10), (12), (13), (14)). However, as a generalisation, it may be said that an increase of the Mn SOD expression/translation under the influence of LPS is reported in most cases, whereas the corresponding effect with regard to Cu/Zn SOD was not observable or was observable only to a substantially less pronounced extent. In most work, even a decrease in Cu/Zn SOD under the influence of LPS is reported (cf. for example (1) to (4), (7), (9), (10), (12) to (14)). The increased formation of SOD in various inflammatory diseases was discussed as part of a protection mechanism against reactive oxygen species (ROS), and SOD was accordingly also used as a therapeutic agent, inter alia for suppression of inflammation (cf. (5)).

The fact that many of the results were obtained with various animal models additionally complicates the picture. Thus, protective effects of LPS administration observed in rats were not verifiable, for example, in cattle (cf. for example (13) and the further literature references mentioned therein). However, there is consensus that SOD plays an important physiological role inter alia in relation to oxygen stress and inflammatory reactions of the body.

Accordingly, SOD was also discussed in relation to sepsis or complications typical of sepsis. The publication (8) or patent application WO 94/22016 A1 linked therewith reports that sepsis patients have raised levels of Mn SOD and that, in the case of Mn SOD levels above a certain cut-off, the risk of developing adult respiratory distress syndrome (ARDS) feared as a complication is considerably increased. Mn SOD was measured immunologically by means of a selective ELISA assay.

On the basis of these results and with the reference to them, the role of SOD in sepsis was subsequently investigated several times, in particular inter alia also in the hope of obtaining early indication of the expected course of a sepsis through the SOD determination (cf. (11), (15) to (17), (19), (20)). In all relevant investigations, however no selective determination of individual SOD types was effected but the SOD enzyme activity was determined generally by methods ((30), (31)) which permit no information about the SOD type to which the enzyme activity found in the sample is attributable. Against the background of the abovementioned basic publication according to (8) or WO 94/22016 A1, however, it had to be assumed that the measured SOD enzyme activity is at least predominantly attributable to increased Mn SOD and possibly additionally EC-SOD values. A specific role of Cu/Zn SOD or particular usefulness of the selective determination of Cu/Zn SOD was not discussed in any of the abovementioned publications in relation to sepsis. It should be noted in this context that the measurement of an enzyme activity cannot be considered to be equivalent to the measurement of the concentration of a biomolecule responsible for the enzymatic effect, or a group of such biomolecules, since the enzyme activity may, for example, be inhibited or enhanced in the samples investigated, without the concentration of the enzyme or of the enzymes necessarily having to change. For SOD, for example, a reduction in the enzyme activity by reactive nitrogen species (RNS) was described (24).

Even in more recent publications which show that the expression of the Cu/Zn SOD gene appears to be controlled by nitric oxide ((14), (18)), no relationship is established between the concentration of the Cu/Zn SOD measurable in a sample of a septic patient and the expected course of a sepsis. The increased Cu/Zn SOD expression is discussed as a protection mechanism.

In view of the clear evidence of Cu/Zn SOD in the baboon sepsis model of the Applicant and in view of the fact that the data to be found in the literature on the occurrence of SOD in sepsis with regard to the role of human SOD in the sepsis process and the diagnostic usefulness of a specific determination of human Cu/Zn SOD in serum or plasma samples of sepsis patients give no reliable indication at all, the Applicant decided, by measuring serum or plasma samples of a larger group of sepsis patients, to check whether there is any relationship between the measurable concentrations of human Cu/Zn SOD in the samples and the documented course of a disease of the patient from whom the investigated samples originated.

The reason for this was the painful deficiency that, in spite of the proven diagnostic sepsis marker procalcitonin introduced in the meantime in clinical practice there is still an urgent need for additional biomarkers for precise sepsis diagnosis and in particular for early sepsis diagnosis, which biomarkers have a diagnostic and especially prognostic potential which can expediently supplement the continuous clinical multiparameter monitoring of patients in intensive care units (e.g. according to APACHE II SCORE) or is optionally even equivalent to such monitoring.

Surprisingly, the measurements of Cu/Zn SOD in serum or plasma samples of patients in intensive care units and emergency care units which were carried out by the Applicant showed that the measured values have an outstanding correlation to the observed course of the disease or course of the sepsis and in particular permit a reliable early assignment of critically ill patients to the groups consisting of the patients who will probably survive and to the patients who will not survive. The quantitative determination of Cu/Zn SOD showed, with remarkable diagnostic sensitivity and specificity not surpassed by any other biochemical markers investigated, the probability of survival of the patients who were delivered to the intensive care unit—as a rule with suspected sepsis or diagnosed sepsis. It therefore permits the identification of a particular risk group of patients among sepsis patients.

Accordingly, the present invention relates, as claimed in claim 1, to a method for the early determination of the risk of mortality of patients in intensive care units and emergency care units, in which the concentration of Cu/Zn superoxide dismutase (Cu/Zn SOD or SOD-1) is determined selectively in a serum or plasma sample of such a patient and—quantitatively or semi-quantitatively—measured concentrations which are above a predetermined cut-off are correlated with a high risk of mortality (a low probability of survival).

Developments of such a method which are advantageous or customary in the technical area form the subject of claims 2 to 12.

Below, the method according to the invention is explained in more detail with reference to results of measurements and graphs illustrating these results of measurements.

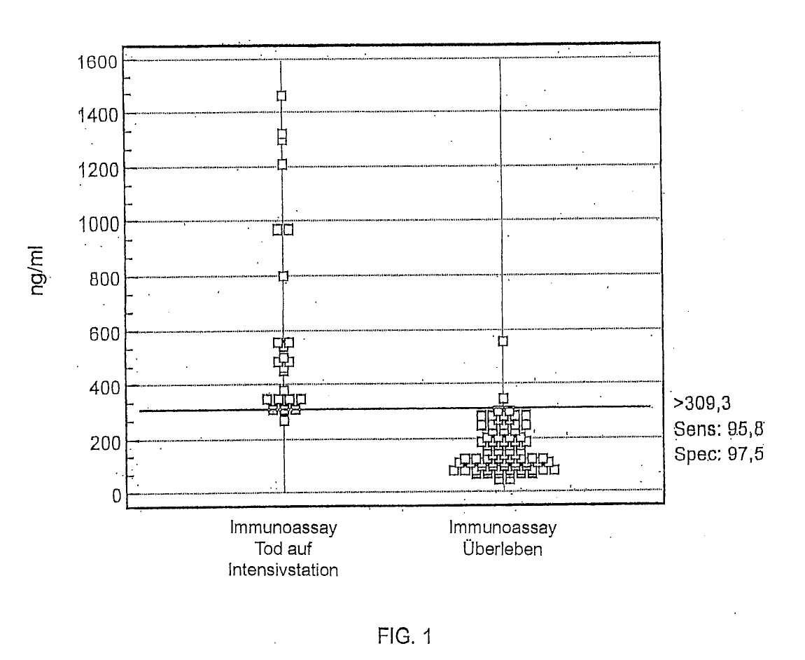

FIG. 1 shows the values of the measurement of the Cu/Zn SOD concentrations for a group of sepsis patients the measurements having been effected with the aid of an enzyme immunoassay specific for Cu/Zn SOD. The samples were assigned to a group of patients expected to die and to a group of surviving patients, taking into account the associated documentation of the course of the disease.

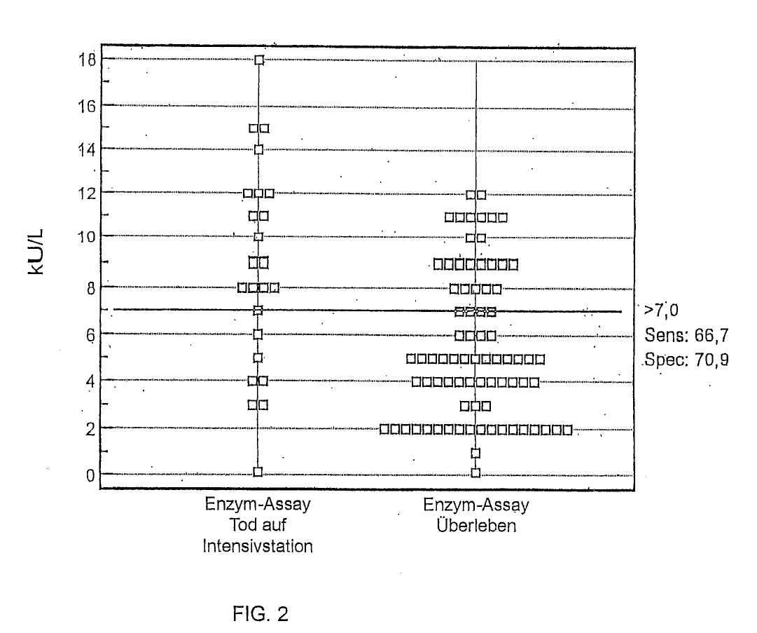

FIG. 2 shows the results of the measurements of the enzymatic SOD activity for the same samples as in FIG. 1, in the same division into patients expected to die and those expected to survive.

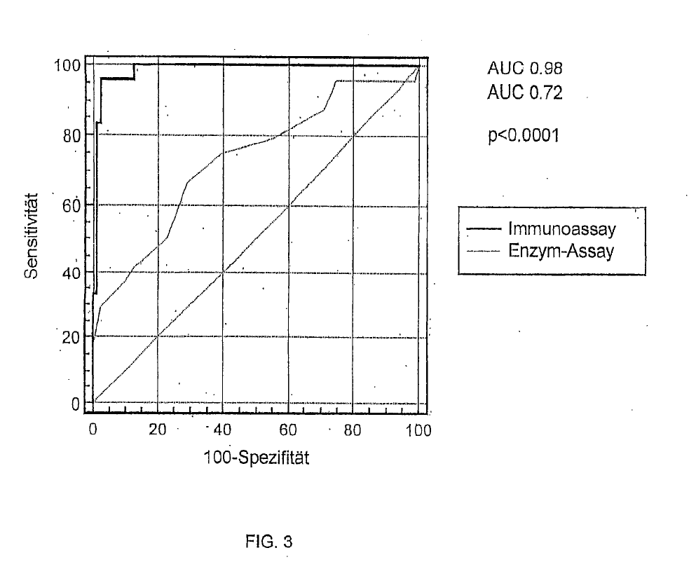

FIG. 3 shows an evaluation of the results of measurements shown in FIGS. 1 and 2, in the form of an ROC plot, the results of the immunochemical measurements of the CU/Zn SOD (solid bold line) being compared with the results of the measurement of the SOD enzyme activity (dotted line).

As is evident from the following examples, the immunochemical measurements of Cu/Zn SOD in plasmas of patients in intensive care units (sepsis patients) were carried out by an ELISA assay (31) commercially available for research purposes, according to the manufacturer's instructions.

However, it is of course also possible to use any other known immunoassays or immunoassays operating according to known principles for determining Cu/Zn SOD, provided that the required specificity and sensitivity are achieved. Examples of such assays are to be found, inter alia, in (2) and patent applications such as EP 217 542 A2, EP 327 337 A2 and WO 90/06513 A1.

In principle, all immunochemical methods suitable for the selective quantitative or at least semi-quantitative determination of Cu/Zn SOD can be used for the measurement.

In a preferred embodiment, the method is carried out as a heterogeneous sandwich immunoassay in which an antibody specific for Cu/Zn SOD is immobilised on an arbitrary solid phase, for example the walls of coated test tubes (e.g. of polystyrene; “coated tubes”; CT) or on microtiter plates, for example, of polystyrene, or on particles, for example magnetic particles, while a further antibody carries a radical which is a directly detectable label or permits a selective link to a label and serves for detecting the sandwich structures formed. Delayed or subsequent immobilization with the use of suitable solid phases is also possible.

In principle, it is possible to employ all marking techniques which can be used in assays of the type described and which include marking with radio isotopes, enzymes and fluorescent, chemoluminescent or bioluminescent labels and directly optically detectable colour markings, such as, for example, gold atoms and dye particles, as are used in particular for so-called point-of-care (POC) or accelerated tests. It is therefore within the scope of the present invention to design the method according to the invention also as an accelerated test.

In the case of heterogeneous sandwich immunoassays, the two antibodies may also have parts of a detection system of the type described below in relation to homogenous assays. The method according to the invention can therefore also be carried out, for example, with the use of a homogenous detection method in which the sandwich complex formed from the two antibodies and Cu/Zn SOD to be detected remain suspended in the liquid phase. In such a case, it is preferable to mark both antibodies with parts of a detection system which permits signal generation or signal triggering when both antibodies are integrated in a single sandwich. Such techniques can be designed in particular as fluorescence amplification or fluorescence extinction detection methods. A particularly preferred method of this type relates to the use of detection reagents to be used in pairs, as described, for example, in U.S. Pat. No. 4,822,733, EP-B1-180 492 or EP-B1-539 477 and the prior art cited therein. They permit a measurement which selectively detects only reaction products which contain both marking components in a single immune complex directly in the reaction mixture. Reference may be made to the technology available under the brands TRACE® (Time Resolved Amplified Cryptate Emission) or KRYPTOR® as an example, which technology implements the teachings of the abovementioned applications.

In the example below, a value of 310 ng/ml was determined as a preferred cut-off for the prognoses “100% mortality risk” or “high probability of survival”. This cut-off can, however, be varied depending on the aim of the prognosis. It should furthermore be noted that the optimal cut-offs mentioned in the example were determined using the commercially available assay according to (31). However, cut-offs are always dependent on the calibration of the immunochemical method used for the determination. If a different immunoassay is used, other absolute cut-offs for the measurable Cu/Zn concentrations may result. An adaptation to the cut-off stated herein but suitable calibration of the specific assay used for the assay according to (31) should, however, always be possible. Where an abovementioned cut-off appears in the patent claims it is to be understood in the abovementioned sense and cannot under patent law be interpreted as an absolute criterion for use of the method according to the invention.

The cut-offs used in a specific clinical environment are therefore calibration-dependent. However, the circumstance presents no problems for the medical practitioner.

### EXPERIMENTAL RESULTS

1. Materials and Methods:

The statistical evaluation of the measurement of the Cu/Zn SOD concentrations in plasmas of patients in intensive care units is described below. Each of the samples measured had been worked up immediately after sampling (venous blood) in a professional manner to obtain an EDTA plasma, and the EDTA plasma samples had then been frozen immediately thereafter at −20° C. The frozen plasmas, like the associated documentation of the course of the disease, were stored for measurement purposes. The thawed samples were used not only for the determinations described below but also in the course of various other determinations which need not be explained in more detail in the present context.

The determination of the Cu/Zn SOD concentrations in the individual plasmas was effected using an ELISA assay commercially available for research purposes (“human Cu/Zn SOD ELISA BMS222”, Bender MedSystems, MedSystems Diagnostic GmbH, Rennweg 95b, A-1030 Vienna, Austria) according to the manufacturer's procedure in the associated manual (32).

The determination of the enzymatic SOD activity, in the samples, carried out for comparative purposes, was effected analogously to (11) by the method according to (30).

For the statistical evaluation of the measurements the corresponding commercially available software (Prism 4.0, Graphpad.com) was used.

2. Measurements of Plasmas of a Group of Patients

2a. Primary Measurements

In order to obtain reference values for the following measurements, the Cu/Zn SOD concentrations in samples of healthy test subjects were measured. The values are scattered considerably in the range from 114.1 ng/ml to 352.1 ng/ml about a median value of 224 ng/ml (standard deviation 49.70 ng/ml). No differences between male and female test subjects were observed.

2b Cu/Zn SOD and the Mortality Risk or the Probability of Survival of Patients in Intensive Care Units Who were Diagnosed with Sepsis, Severe Sepsis and Septic Shock

103 plasma samples of a bank available to the applicant and containing samples of sepsis patients (diagnosis: sepsis, severe sepsis and septic shock) from various intensive care units were used for the following measurements. The blood samples were taken within the first 48 h after admission to the intensive care unit.

The measurements of the Cu/Zn SOD concentrations and the evaluation of the measurements were effected as explained. In view of the fact that the determination of Mn SOD is proposed in (8) in relation to sepsis or the development of ARDS, and, based on this, the determination of the SOD enzyme activity in samples of sepsis patients is discussed as a prognosis marker in (11), the measured values obtained for Cu/Zn SOD and their prognostic significance according to the present invention are compared with the results which are obtained for the same samples in a determination of the SOD enzyme activity (kU/L) by a method according to (30) which is used in (11).

The results of the two methods of measurements are shown in FIGS. 1, 2 and 3. On the basis of a grouping of the samples investigated into those of patients expected to die (79 patients) and of survivors (24 patients), the value of 310 ng/ml was determined as a suitable cut-off and was used for the evaluation.

A comparison of FIGS. 1 and 2 shows that an immunochemical determination of the Cu/Zn SOD concentrations in the plasma of patients in intensive care units (sepsis patients) cannot be considered to be equivalent to a determination of an SOD enzyme activity in the same samples, since qualitatively very different results are obtained.

Also evaluated were the results of the measurements of the Cu/Zn SOD concentrations and the measurements of the enzymatic activity carried out for comparative purposes by means of so-called ROC curves (receiver operating characteristic plots) which express the sensitivities and specificities of the Cu/Zn SOD determination for the group of patients in relation to one another. The sensitivity was calculated as the proportion of correctly recognised patients who subsequently died, based on all subsequent deaths. The specificity was calculated as the proportion of correctly recognised survivors, based on the total number of the survivors.

In the case of ROC curves, the area between the relevant curve and a straight line at an angle of 45° (“area under the ROC function” or “area under the curve”, AUC) can be taken as a characteristic for the statistical relevance of a determination. The larger this area the higher is the diagnostic or prognostic significance.

In the case of an ROC evaluation of the values according to FIGS. 1 and 2, it is found, according to FIG. 3, that the specific immunochemical determination of the Cu/Zn SOD (bold solid line) gives very much more informative and significant results than the determination of the SOD enzyme activity (dotted line) in the same samples.

On the basis of the abovementioned results of the statistical evaluation of the measurement of Cu/Zn SOD in plasmas of patients in intensive care units, a new possibility of obtaining a rapid prediction of the chances of survival of patients who are delivered to an intensive care unit by measurement of the concentration of this biomarker Cu/Zn SOD in said patients surprisingly opens up. Such a measurement, in particular in combination with a measurement of procalcitonin, is expedient for sepsis diagnosis. Patients having high Cu/Zn SOD concentrations then constitute a particular risk group among the sepsis patients, who justify particular treatment and therapeutic intervention. In this context, for example, it should be pointed out that activated protein C (Drotrecogin or Xigris from Eli Lilly) has recently been approved in the USA for the therapy of sepsis, but is applicable only to that part of the population of sepsis patients for which there is a high risk of mortality. The question as to how this part of the population can be correctly determined therefore proves to be a challenge. The use of APACHE II SCORES envisaged in the absence of alternatives has been the subject of controversy in the literature (35), (36), (37). The availability of the prognosis marker Cu/Zn SOD as an additional or alternative instrument for identifying high-risk sepsis patients is an important advance in this context.

### LIST OF REFERENCES

1. Michael A. Hass et al., The Effect of Bacterial Endotoxin on Synthesis of (Cu,Zn) Superoxide Dismutase in Lungs of Oxygen-exposed Rats, in: J. Biol. Chem. 257, 9379-9383, 1982
2. K. Asayama et al., Selective induction of manganous superoxide dismutase in human monocytes, in: Am. J. Physiol. 249, C393-C397, 1985
3 Jawaid Iqbal et al., Endotoxin increases lung Cu,Zn superoxide dismutase mRNA: O₂ raises enzyme synthesis, in: Am. J. Physiol 257, L61-L64, 1989
4. Gary A. Visner et al., Regulation of Manganese Superoxide Dismutase by Lipopolysaccharide, Interleukin-1, and Tumor Necrosis Factor, in: J. Biol. Chem. 265, 2856-2864, 1990
5. Marian Gorecki et al., Recombinant Human Superoxide Dismutases: Production and Potential Therapeutical Uses, in: Free Rad. Res. Comms., Vols. 12-13, 401-410, 1991
6. Xue-Jun Kong et al., Regulation of Cu,Zn-Superoxide Dismutase in Bovine Pulmonary Artery Endothelial Cells, in: J. Cell. Physiol., 153:491-497 (1992)
7. Lester S. Gibbs et al., Mn and Cu/Zn SOD Expression in Cells from LPS-sensitive and LPS-resistant Mice, in: Free Rad. Biol. Med. Vol. 12, 107-11, 1992
8. Jonathan A. Leff et al., Serum Antioxidants as predictors of adult respiratory distress syndrome in patients with sepsis, in: Lancet 1993; 341:777-780
9. Kenji Mokuno et al., Induction of Manganese Superoxide Dismutase by Cytokines and Lipopolysaccharides in Cultured Mouse Astrocytes, in: J. Neurochem. 63, 612-616 (1994)
10. Reishi A B E et al., Lipopolysacharide Induces Manganese Superoxide Dismutase in the Rat Pancreas; Its Role in Caerulin Pancreatitis, in: Biochem. Biophys. Res. Comm., Vol. 217, No. 3, 1216-1222, 1995
11. Ann Warner et al., Prognostic Role of Antioxidant Enzymes in Sepsis: Preliminary Assessment, in: Clin. Chem. 41/6, 867-871 (1995)
12. Bidyut Ghosh et al., Tissue Differences in Antioxidant Enzyme Gene Expression in Response to Endotoxin, in: Free Rad. Biol. Med., Vol. 21, No. 4, 533-540, 1996
13. Mary Leach et al., Decline in the expression of copper/zinc superoxide dismutase in the kidney of rats with endotoxin shock: Effects of the superoxide anion radical scavenger, tempol, on organ injury, in: Br. J. Pharmacol., 125, 817-825 (1998)
14. Stefan Frank et al., Identification of copper/zinc superoxide dismutase as a novel nitric oxide-regulated gene in rat glomerular mesangial cells and kidneys of endotoxemic rats, in: FASEB J. 13, 869-882 (1999)
15. Seema et al., Serum TNF-Alpha and Free Radical Scavengers in Neonatal Septicemia, in: Indian J Pediatr. 1999; 66:511-516
16. N. K. Dubey et al., Free Oxygen radicals in acute renal failure, in: Indian Pediatrics 2000; 37:153-158
17. Simmi Kharb et al., Role of Oxygen Free Radicals in Shock; JAPI 2000; 48:956-957
18. Stefan Frank et al., Identification of copper/zinc dismutase as a nitric oxide-regulated gene in human (HaCaT) keratinocytes. Implications for keratinocyte proliferation, in: Biochem. J. (2000) 346, 719-728
19. Sanjay Batra et al., Alterations in antioxidant status during neonatal sepsis, in: Ann. Trop. Paediatrics (2000) 20, 27-33
20. P. Bela et al., Oxidative stress status: possible guideline for clinical management of critically ill patients; in: Panminerva Med 2001, 43(1):27-31
21. Yasuda M. et al., Prognostic significance of serum superoxide dismutase activity in patients with gastric cancer, in: Gastric Cancer 2002; 5(3): 148-53
22. Taysi S, et al., Lipid peroxidation, some extracellular antioxidants, and antioxidant enzymes in serum of patients with rheumatoid arthritis, in: Rheumatol Int 2002 21(5): 200-204;
23. Taysi S et al., Serum oxidant/antioxidant status of patients with systemic lupus erythematosus, Clin Chem Lab Med 2002, 40(7):684-688
24. John M. Lawler et al., Specificity of antioxidant enzyme inhibition in skeletal muscle to reactive nitrogen species donors, in: Biochem. Biophys. Res. Commun. 294 (2002) 1093-1100
25. Paolo Mondola et al., The Cu,Zn superoxide dismutase in neuroblastoma SK-N-BE cells is exported by a microvesicles dependent pathway, in: Mol. Brain Res. 110 (2003) 45-51
26. Moshe Marikovsky et al., Cu/Zn Superoxide Dismutase Plays Important Role in Immune Response; J. Immunol., 2003, 170:2993-3001
27. Barra D., et al., The complete amino acid sequence of human Cu/Zn superoxide dismutase; FEBS Letters 120, 53-56 (1980)
28. Beck Y. et al., Human Mn superoxide dismutase cDNA sequence; Nucleic Acids Res. 16:6224
29. Hjalmarsson K. et al., Isolation and sequence of complementary DNA encoding human extracellular superoxide dismutase; Proc. Natl. Acad, Sci. U.S.A. 84:6340-6344 (1987)
30. Joseph B Z et al., Activities of SODs and NADPH oxidase in neutrophils obtained from asthmatic and normal donors; Inflammation 1993; 174:331-336
31. Mishra H P et al., The role of superoxide anion in the antioxidation of the epinephrine and simple assay for superoxide dismutase; J. Biol. Chem. 1971; 247:3170-3175
32. Product Information and Manual: human Cu/Zn SOD ELISA BMS222, Bender MedSystems, MedSystems Diagnostics GmbH, Rennweg 95b, A-1030 Vienna, Austria (Mar. 9, 1997)
33. G. Pilz et al., Krankenpflege-Journal 29 (1991), S.483-492
34. Niels C. Riedemann et al., The enigma of sepsis, J. Clin. Invest. 112:460-467 (2003)
35. Manns Bj et al., An economic evalutation of activated protein C treatment for severe sepsis, N Engl J Med 2002; 347:993-1000
36. Warren H S et al., Risks and benefits of activated protein C treatment for severe sepsis, N Engl J Med 2002; 347:1027-1030
37. Siegel J P, Assessing the use of activated protein C in the treatment of severe Sepsis, N Engl J Med 2002; 347:1030-1034

1. A method for the early determination of the risk of mortality of patients in intensive care units or emergency care units, wherein the concentration of Cu/Zn superoxide dismutase (Cu/Zn SOD or SOD-1) is selectively determined in a serum or plasma sample of such a patient, and concentrations which are above a predetermined cut-off are correlated with a high risk of mortality.

2. The method as claimed in claim 1 wherein the patients are patients in intensive care units for whom the clinical diagnosis is sepsis, severe sepsis or septic shock.

3. The method as claimed in claim 1 or 2, wherein the method for the determination of the Cu/Zn SOD concentrations is an immunochemical assay method selective for Cu/Zn SOD.

4. The method as claimed in claim 3, wherein the selective immunochemical determination method is a ligand binding assay of the competitive type or sandwich type.

5. The method as claimed in any of claims 1 to 4, wherein the correlation between the Cu/Zn SOD concentration present in the serum or plasma sample and the cut-off is established by a quantitative or semi-quantitative concentration determination.

6. The method as claimed in claim 4 or 5, wherein the ligand binding assay is a homogeneous or heterogeneous immunoassay of the sandwich type, in which at least one marked monoclonal or polyclonal antibody is used for detecting Cu/Zn SOD and the marking is selected from radioisotope, fluorescence, chemiluminescence, enzyme and direct optically detectable dye particles.

7. The method as claimed in any of claims 1 to 6, wherein a value of 310 ng/ml or more is chosen as the optimal cut-off for the measured Cu/Zn SOD concentration.

8. The method as claimed in any of claims 1 to 7, which is carried out as part of a multiparameter determination in which a quantitative or qualitative determination of at least one further sepsis prognosis parameter is effected at the same time.

9. The method as claimed in claim 8, wherein at least one further parameter which is selected from the group which consists of procalcitonin, CA 19-9, CA 125, S100B, S100A proteins, soluble cytokeratin fragments, in particular CYFRA 21, TPS and/or soluble cytokeratin-1 fragments (sCY1F), the peptides inflammin, CHP, LASP-1, GNAT, mutarotase, CPS 1 and the peptide prohormones proANP, proBNP, proADM and the C-reactive protein (CRP) is determined as part of the multiparameter determination in addition to Cu/Zn SOD.

10. The method as claimed in claim 8 or 9, wherein the multiparameter determination is effected as a simultaneous determination by means of a chip technology measuring apparatus or an immunochromatographic measuring apparatus.

11. The method as claimed in claim 10, wherein the evaluation of the complex result of the measurement obtained using the measuring apparatus is effected with the aid of a computer program.

12. The method as claimed in any of claims 1 to 5, which is carried out as an immunochromatographic point-of-care method (accelerated test).
